(19) United States
(12) Patent Application Publication
(10) Pub. No.: US 2002/0177186 A1
(43) Pub. Date: Nov. 28, 2002

(54) METHOD FOR THE REGULATION OF PROTEIN BIOSYNTHESIS

(76) Inventor: JOEL STERNHEIMER, RESIDENCE (XP)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 09/320,637

(22) Filed: May 26, 1999

Related U.S. Application Data
(63) Continuation-in-part of application No. 08/347,353, filed on Dec. 1, 1994, now abandoned.
Application No. 08/347,353 is a 371 of international application No. PCT/FR92/00524, filed on Jun. 2, 1993.

(30) Foreign Application Priority Data
Jun. 4, 1992 (FR) 92-06765

(51) Int. Cl. C12P 21/06; A01N 37/18; A01N 43/04

(52) U.S. Cl. 435/69.1; 514/2; 514/44

(57) ABSTRACT

There is provided a method for determining the musical notes associated with an amino acid sequence, the musical periods of the sequence, the lengths of the notes, and the tone quality of the notes through the retroaction of the whole set of amino acids and using that information to regulate the biosynthesis of the protein. The amino acids that build a protein emit a signal of quantum nature at a certain frequency. Following the properties of this signal, the frequency is transposed into a musical note. This discovery has numerous applications since one can then deduce from the amino acid sequence of a protein a sequence of notes composing the melody that will act to stimulate or inhibit its synthesis inside an organism, wherefrom one can in addition delimit its biological functions.

### CROSS-REFERENCE TO RELATED APPLICATION

This application is a continuation-in-part of U.S. patent application, Ser. No. 08/347,353 filed Dec. 1, 1994.

### BACKGROUND OF THE INVENTION

The present invention is directed to a method of regulating protein biosynthesis. More particularly, the invention is directed to a method for epigenetic regulation of in situ protein biosynthesis and its use in agronomy and health.

Demonstration of the musical properties of elementary particles suggests an important role for the scale at which the phenomena happen. (J. Sternheimer, C. R. Acad. Sc. Paris 297, 829, 1983). For example, it is known that the physical existence of quantum waves associated to particles propagate themselves not only in space-time, but also in that scale dimension, thus linking together successive levels of the organization of matter. (J. Sternheimer, Colloque International “Louis de Broglie, Physician et Penseur”, Ancienne Ecole Polytechnique, Paris, Nov. 5-6, 1987). These waves allow an action of one scale onto the other, between phenomena that are similar enough to constitute, in a mathematically well-defined sense, harmonics of a common fundamental tone. (See J. Sternheimer, Ondes d'e'chelle [scaling waves], I. Partie Physique; II. Partie Biologique. Filed at Academie des Sciences (Paris) 1992 under seal no. 17064).

The theoretical reasons for the existence of scaling waves makes them appear as a universal phenomenon whose function is at first to ensure coherence between the different scales of a quantum system, and that especially takes shape and can be described in the process of protein biosynthesis. The peptidic chain elongation effectively results from the sequential addition of amino acids that have been brought onto the ribosome by specific transfer RNAs (tRNAs). When an amino acid, initially in a free state, comes to affix itself to its tRNA, it is stabilized with respect to thermal agitation —while keeping a relative autonomy because it is linked to the tRNA by only one degree of freedom—for its de Broglie wavelength to reach the order of magnitude of its size. This stabilization gives the amino acid wave properties.

Interference between the scaling wave associated to the amino acid and those similarly produced by the other amino acids, results in a synchronization, after a very short period of time (which can be evaluated to be about 10^{-12.5} second), of the proper frequencies associated with these amino acids according to one and same musical scale, which more precisely depends upon the transfer RNA population. However, to within the approximation of the chromatic tempered scale, this scale appears universal due to the very peculiar distribution of amino acid masses which is already very close to it.

The scaling wave phenomenon appears in a more explicit way when the amino acid carried by its tRNA fixes itself onto the ribosome. It is at this moment that the stabilization with respect to thermal agitation becomes such that the wavelength of the amino acid outgrows its size by a full order of magnitude. The scaling wave which then emits interferes, at the scale of the protein in formation, with similar waves previously emitted by the other amino acids. This interference draws constraints of a musical type for the temporal succession of the proper frequencies associated to these waves, so that the scaling waves continue their itinerary and insure coherence and communication between different levels of the organism. For example, the succession of these waves minimizes the dissonance (harmonic distance) and the frequency gaps (represented by melodic distance) between successive amino acids. Additional properties imply the existence of periods of minimization of harmonic distances showing punctuations in the temporal succession of frequencies which other levels will complete with correlations all the more rich and marked that they themselves are more numerous to influence the protein synthesis. The result is the prediction that proteins possess, in the very succession of the proper quantum frequencies associated to the sequence of their amino acids, ‘musical’ properties all the more clear and elaborate that their biosynthesis is more sensitive to epigenetic factors in general. Conversely, it must be possible to act epigenetically, in a specific way for each protein onto that biosynthesis.

The observation of protein sequences confirms that all proteins possess musical properties in the sequence of their amino acids and these properties are all the more developed that those proteins are, in a general way, more epigenetically sensitive. (Data from M. O. Dayhoff, Atlas of protein sequence and structure, volume 5 and supplements, N.B.R.F. (Washington) 1972-78). In addition, the acoustic transposition of the series of proper frequencies corresponding to the production of scaling waves in phase with the elongation of a given protein,.shows a stimulating action onto the biosynthesis of this protein in vivo, and in a correlative way it has an inhibiting action for scaling waves in phase opposition.

In the case of animals having a nervous system the sound wave is transformed into electromagnetic impulses of the same shape and frequency right from the starting point of the auditory nerve. These impulses, by virtue of the scale invariance of scaling wave equations applied to the photon (which generalize Maxwell's equations), have a direct action, by scale resonance, on their quantum transpositions. Because the squared quantum amplitudes are proportional to the number of proteins that are simultaneously synthesized, the resonance phenomenon results, in the case of scaling waves in phase, in an increase of the rate of synthesis, as well as a regulation of its rhythm, and in the case of scaling waves in phase opposition, in a reduction of this rate. (cf. P. Buser and M. Imbert, Audition, Hermann èditeur, Paris, 1987). Among plants, the sensitivity to sounds is visible through interferometry and the scaling waves behave theoretically in a similar way.

The solution to the scaling wave equation, which effectively shows the existence of scaling waves having a range close to Avogadro number, anticipates similar properties for the scaling waves drawn from the spatial distribution of amino acids (whose de Broglie wavelength is then comparable to their size) inside the protein after it has been synthesized. The solution then provides a range approximating the square root of that number. The observation of their tertiary structures confirms the existence of harmonies within vibratory frequencies of amino acids spatially nearby inside proteins (and especially at their surface, as can be expected from their wavelength). An appreciable stabilization of the effects obtained with the use of the musical transpositions is then observed using colored transpositions of these spatially distributed frequencies.

The present invention is drawn from these observations.

### SUMMARY OF THE INVENTION

The method of the invention comprises determining the musical notes associated with an amino acid sequence, the musical periods of the sequence, the lengths of the notes, and the tone quality of the notes through the retroaction of the amino acids and using that information to regulate the biosynthesis of the protein.

Stated in another way, the amino acids which build a protein emit a signal of quantum nature at a certain frequency. Following the properties of this signal the frequency is transposed into a musical note in such way that playing back the melody of a protein will stimulate or inhibit its synthesis. This discovery has numerous applications since deduction of the amino acid sequence of a protein provides a sequence of notes composing the melody which will act on its synthesis inside an organism. Thus, by diffusing to a plant the music of a protein which plays an important role in flowering, more flowers are produced.

Stated more scientifically, the method of this invention uses the regulating action on the biosynthesis of proteins by scale resonance of transpositions into sound of temporal sequences of quantum vibrations associated with their elongation. This action may be an increase of the rate of synthesis or a reduction of this rate, depending upon whether the modulation of the vibration frequencies used is in phase with, or in phase opposition to the elongation. This is true for the quantum vibrations as well as for their transposition into sound. The result is further stabilized by the actions, again through scale resonance, of colored light transpositions of grouped quantum vibrations arising from the spatial conformation of proteins issued from this elongation.

This method applies in a specific way to every protein of known structure. Its use is all the more appropriate when the synthesis of this protein is even more dependent upon epigenetic factors, that is to say external to the DNA of the system to which it belongs, and especially in the present case, upon acoustic and electromagnetic factors. In addition, the method uses the determination of metabolic agonisms and antagonisms of these proteins due to scale resonance phenomena naturally associated with their biosynthesis. The characterization of these proteins in their associated metabolic subsets is another feature of the present invention.

The identification of proteins designed to be regulated as part of a given application includes other criteria a correspondence between acoustic and electromagnetic phenomena or which effects can be observed on living beings and the transposed proteic sequences.

### BRIEF DESCRIPTION OF THE INVENTION

Certain features and advantages will be evidence from the drawings when considered in conjunction with the accompanying drawing in which:

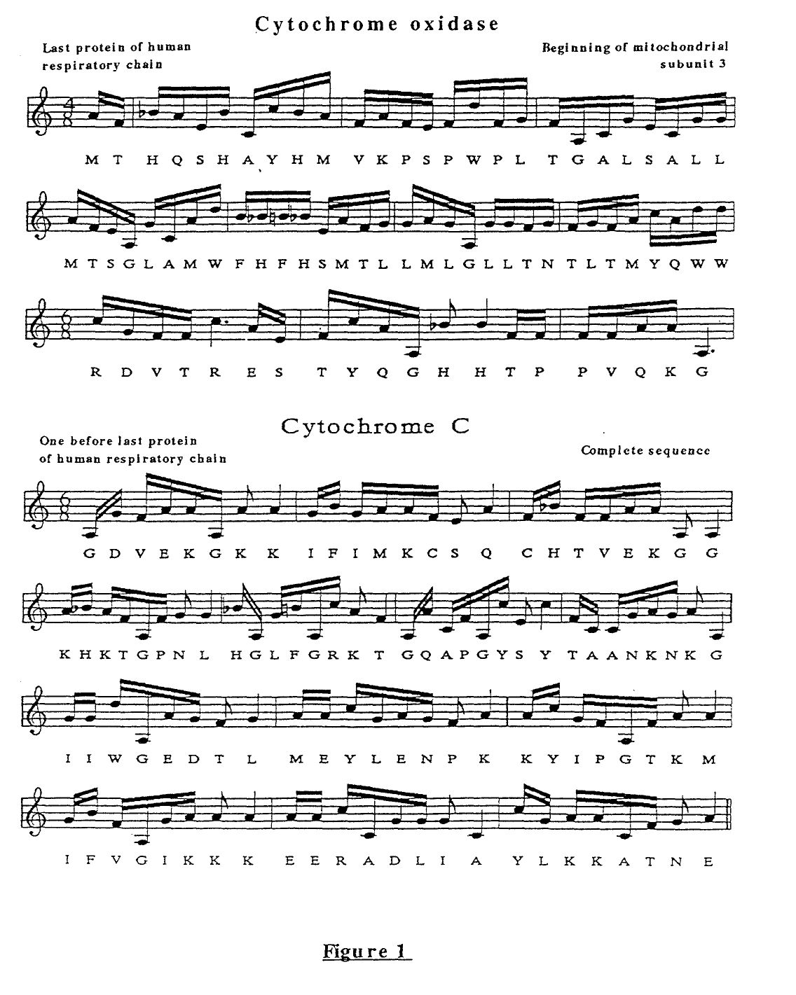

FIG. 1 shows the musical scale cytochrome oxidaze and cytochrome C;

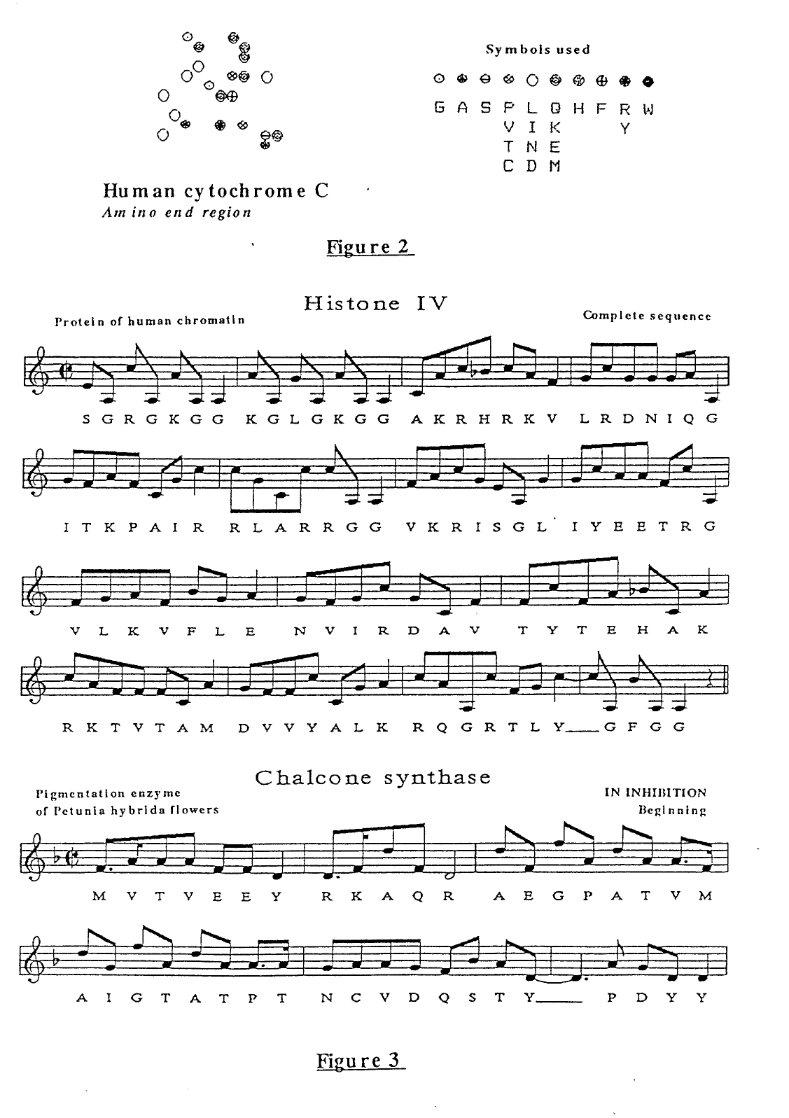

FIG. 2 shows the cytochrome C humain region for amino-terminal and legends;

FIG. 3 shows Hystone IV and chalconesynthase; and

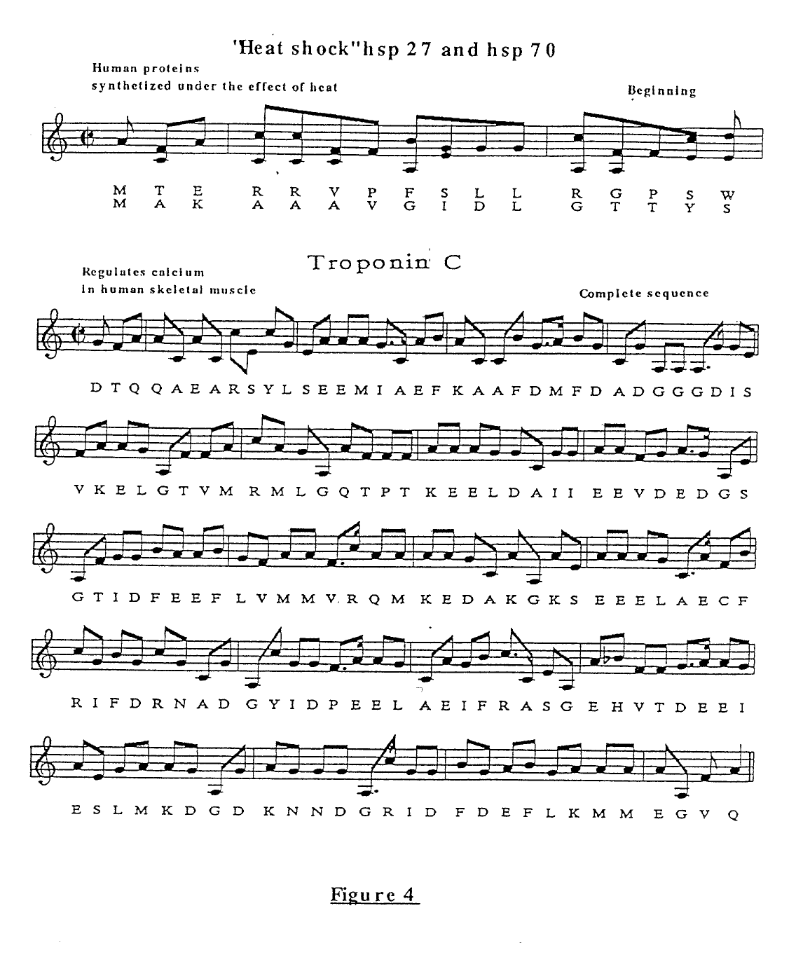

FIG. 4 shows “heat shock” HSP 27 Ethsp 70 and Troponinec.

### DETAILED DESCRIPTION OF THE INVENTION

The present invention will now be described more fully hereinafter with reference to the accompanying drawings, in which preferred embodiments of the invention are shown. This invention may, however, be embodied in many different forms and should not be construed as limited to the embodiments set forth herein; rather, these embodiments are provided so that this disclosure will be thorough and complete, and will convey the scope of the invention to those skilled in the art.

There is provided a method of regulating protein synthesis in situ, using a musical sequence corresponding to the amino acid sequence of a protein through the decoding and transposition into sound of a temporal series of quantum vibrations associated with the elongation of the amino acid chain of the protein. The method of regulating protein synthesis in situ requires at least the following steps: the sequence of musical notes is determined; the period appearing in the molecule is determined; the period is rectified, if necessary; the rhythmic style is checked through the distribution of the bases of DNA; and the tone quality is determined.

Determining The Sequence Of Musical Notes. The sequent of music notes associated with the amino acid chain of a protein is determined by associating a musical note with each amino acid. More specifically, within the approximation of the tempered scale a universal code for the stimulation of protein synthesis is obtained. That code is:

Gly=low A; Ala=C; Ser=E; Pro Val, Thr, Cys=F; Leu, Ile, Asn, Asp=G; Gln, Lys, Glu, Met=A; His=B flat; Phe, as well as SeC=B; Arg, Tyr=sharp C; Trp=sharp D

which are deduced from the notes of the code by taking the notes of the chromatic tempered scale which are symmetrical to those of said keynotes with respect to central G.

There is another code for inhibition, which is deduced from the preceding code by symmetrization of the logarithms of the frequencies around their central value:

Trp=C; Arg, Tyr=D; Phe, SeC=E flat; His=E; Gln Lys, Glu Met=F; Leu, Ile, Asn, Asp=G; Pro, Val, Thr, Cys=A; Ser=B flat; Ala=sharp D; Gly=sharp F

that are deduced from the notes of the code by taking the notes of the chromatic tempered scale which are symmetrical to those of said keynotes with respect to central G.

The application of the universal code results in scaling waves respectively in phase with and in phase opposition to those taking place during the synthesis process. The term “universal code” means that this code is identical for all proteins to within the approximation of the tempered scale; the low A, for a central frequency located 76 octaves below the centre of gravity of the initial frequencies of leucine, isoleucine, and asparagine, is at 220 Hz. The expression of harmonic distance given above extends the definition suggested by Y. Hellegouarch in C. R. Math. Rep. Acad. Sci. Canada, Volume 4, Page 227, 1982. The exact values of the frequencies depend on the proportions of the groups of the above-mentioned amino acids among the transfer RNA population surrounding the protein biosynthesis.

Determination of Frequency. The next step is to derive the frequency of each of the notes. The following code is derived in the following manner, which also optionally enables to give a more precise frequency value to each note. The frequency of the musical notes is calculated from the frequencies of amino acids in their free state (proportional to their masses) by minimizing the global harmonic distance Σij Pᵢ Pⱼ logsup (pi, qj) calculated for all possible pairs of notes, (pi/qj) being the harmonic intervals globally the closest to the corresponding proper frequency ratios. Their respective proportions Pᵢ, Pⱼ in the environing population of transfer RNAs are taken into account. While respecting the condition δf<Δf/2 where δf is the displacement of the initial frequency towards its synchronized value and Δf is the interval between the two successive synchronized frequencies of the obtained scale, which encompass this initial frequency. The resulting frequency is then transposed into the field of audible frequencies. See, method described in the French patent number 8302122.

Determination Of The Musical Period. Once the frequency of each musical note is determined, the musical period is determined by identifying similar series of musical notes. The existence of musical periods results directly from that of scaling waves.

An indication is given by the presence of obvious cadences producing punctuations in the musical development. Obvious cadences include such cadences as GG, F-S. That is to say, F closely followed by S, as well as the cadence ending the signal peptide when it is present, for stimulation; series of R or Y, for inhibition; exceptionally, relative pauses induced by harmonic variations which would otherwise be too straight; and in all cases, cadences expressing the return to the tonic note.

The similar passages are then determined. One method of determination is by the direct repetition of notes. When this method is used the period is given by a simple calculation of autocorrelations of notes. More specifically, by minimizing the frequency differences between notes by the number that minimizes the average on the protein of melodic distances between notes located an integer number of intervals apart.

A second method is to determine the melodic movements of the musical notes. The period is calculated by autocorrelations of signatures—or frequency variation signs—from one note to the next. More specifically, the period is determined by calculating autocorrelations of the melodic distances from one note to the other, the distances being counted with their sign, i.e., multiplied by the corresponding signatures; or even more finely, by the number which minimizes the average on the protein of step by step melodic distances variations, to within an integer number of intervals apart. The repetition of the melodic contours are processed by a calculation of autocorrelations of pairs, or even better, of triplets of signatures.

A third method of determining the period of the musical notes is by the logic of the harmonic movement that reproduces the notes or the melodic movement to the nearest simple harmonic transposition. The period is then given by the number that minimizes the average on the protein of harmonic distances between notes located an integer number of intervals apart.

Sometimes when an “alignment” of similar sequences is present, the period appears in the additions or in the deletions of certain of the sequences. The result gives a melodically and harmonically coherent progression. To do that, account is taken of the fact that the last notes of each period or member of phrase—usually the second half, and more particularly the last note—as well as those situated on the strong beat are the most important for this progression. The final result is the most significant respecting the whole of these criteria. These different elements are balanced according to their relative importance in the protein, and especially the harmonic and melodic distance by the square of the ratio of their normalized standard deviations. There is usually one that is distinctly more significant than the others.

Cases similar to allosteria nevertheless exist, and have a biological meaning (stimulation or inhibition by such molecule or such other one during the metabolism), but influence more frequently the position of the measure bars than the period. It is noted that metabolic function is different according to the context, for instance, CG rich or AT rich; the measure bars depending upon the composition of the DNA, as the “Christmas trees” that can be seen during certain syntheses clearly displayed (cf. B. Alberts and al., Molecular biology of the cell, 2nd edition, Garland Publ. Co. 1989, page 539).

Determining The Lengths Of Musical Notes. If necessary, the period is rectified so that the melodic passages that repeat or follow one another can be found in the same place inside the measure. From this rectification the individual lengths of the musical notes are deduced. This operation of adjusting the phrasing to the measure is comparable to the well known phenomenon of lengthening the vowels of a sung text.

In practice, the operations described above can be performed most easily with a keyboard, such as a Casio™ equipped with a “one key play” device, or with a computer programmed especially for that purpose with stored sequence of musical notes and where the sequence of notes can be played. However, some precautions are required. Prudence implies, among other things, to decode the same molecule or a musically similar molecule, in the direction of inhibition (or in any case in the direction opposite from the initial one), taking into account the fact that molecules very often have a preferential decoding direction. It is often the case that pairs of molecules that sensibly exert the same function find one pair being more musical in inhibition and the other one in stimulation.

Checking Rhythmic Style Through The Distribution Of The Bases of DNA. When the molecule is musical enough, the period of autocorrelations corresponds to that of the protein. The autocorrelations determine in principle the measure bars, the ranks of base triplets—or more precisely of bases in third position in these triplets—for which the peaks of autocorrelation are the highest, corresponding to the most accentuated notes. By referring to codon sage, in comparison with known molecules (already decoded, or more regular and thus raising less difficulties) having the same supposed rhythmic style; the style of musical rhythm (which by constraining the accentuation of notes, influences the choice of bases in third position) determining the codon usage. Molecules of the same style must therefore have the same codon usage. If necessary, the decoding of some passages is corrected.

Determining The Tone Quality. Tone quality is, in principle, different for every molecule and for every distribution of musical notes. In theory, tone quality mainly depends upon the molecule itself but it also depends upon all the levels of the organism which retroact on the harmonic structure of amino acid vibrations. The tone quality of the musical sequence is determined by comparing the repartition of the music sequence of the amino acid chain to the average repartition of those notes of the whole of the protein to determine which harmonics must be raised or lowered. The term “tone quality” or timbre is characterized by the harmonic structure of a note and more precisely by the variation of harmonic structure over a given note.

A first approach is given by adjusting the distribution of molecule notes to the theoretical graph of that distribution. The distribution is deduced from the scaling wave equation. The distribution also corresponds to what can be observed in average, on the whole of proteins. This adjustment to the tone quality requires determination of which harmonics are amplified and which are softened in the wanted tone. See, French Patent No. 8302122. The closest tone quality is then selected in a palette of given ones. For example, a voice memory or as one can already find included in many expanders and musical softwares. To distinguish more precisely between three situations: (1) distribution of notes constant along the molecule to provide a relatively fixed harmonic structure; (2) straight distribution changes to provide different successive tones of instrument, for instance cytochrome C with several organ registers; and (3) progressive distribution change which then reproduces the time evolution of the harmonic structure of one note, for example, myosin, where this evolution indicates a timbre of trumpet.

Apart from this, determining the tempo gives no real problem to the technician because it normally follows from the rhythmic style. It is generally all the faster that there are important redundancies in the proteic sequence, as it is the case for fibrous proteins.

Determining The Colors. Optionally, the colors are determined by applying the universal code. The color is deduced from vibration frequencies of individual amino acids through the formula (drawn from scaling wave theory): ν˜ν₀ Argch (e (ƒ/ƒ₀) Logch 1), where (ƒ, ƒ₀ represent the proper quantum frequencies associated with aminoacids as previously, and ν, ν₀ those of colors, the index 0 showing central values. This gives the following code relating to the stabilization of proteins synthesized in situ (the code related to the stabilization of their inhibition is deduced as in section 1 by symmetrization of the logarithms of frequencies with respect to the central lemon yellow):

Gly=dark red: Ala=bright red: Ser=orange; Pro, Val. Thr, Cys=ochre; Leu, Ile, Asn, Asp=lemon yellow; Gln, Glu, Lys, Met=green; His=emerald: Phe=blue; Arg, Tyr=indigo; Trp=purple,

these frequencies then being moved towards red or purple according to the global repartition of the molecule frequencies in a way similar to the description for tone quality as above. The spatial position of colors is the same as those of the amino acids in the tridimensional spatial representation of the molecules.

Several examples are set forth below to illustrate the invention and the manner in which it is carried out. In these examples as well as in the figures, the one-letter notation for amino acids: Gly=G; Ala=A; Ser=S; Pro, Val, Thr, Cys=P, V, T, C respectively; Leu, Ile, Asn, Asp=L, I, N, D; Gln, Glu, Lys, Met=Q, E, K, M; His=H; Phe=F; Arg, Tyr=R, Y; Trp=W is used.

### EXAMPLE 1

This example illustrates decoding a protein that is regular from beginning to end. Cytochrome C provides a constant deletion of eight amino acids (sometimes seven) among animal proteins when compared to plants. Observing the autocorrelations of musical notes and melodic contours confirmed the value of the musical period.

The occurrences of the same note was counted and the same direction of pitch variation occurred three times in a row (the same triplet of signatures), which was distant from an integer number k of musical notes. The following result was obtained:

Values of k 1 2 3 4 5 6 7 8 9 10 11 12

Note autocorrelations 19 15 15 20 19 15 17 21 14 17 18 13

Melodic contour autocorr. 1 7 4 6 5 10 8 13 5 4 4 4

Total 20 22 19 26 24 25 25 34 9 21 22 17

the peak at k=8 being worth about 2.5 standard deviations (as compared to its expectation value 22.3±4.7 determined from the repartition of notes of the molecule). The significance of this peak was reinforced when using melodic distances.

The peak outgrew distinctly 3 standard deviations when the autocorrelations of melodic intervals were included by taking as a definition of the melodic distance between two notes, the absolute value of the difference of the ordinal ranks of their tempered frequencies arranged in ascending order. This definition is derived from the usual nomenclature: second, third, etc., for the notes of a musical mode. The secondary peak at k=7 then became slightly significant, corresponding to the relative stretching of the seventh note which tended to precede the return to the tonic; whereas, the one at k=4 was reinforced when harmonic distances were used to spatial foldings of the molecule.

The observation of the cadences also confirmed this value, as well as that of the internal similarities. The last five notes of the first, second and third group of eight produced together an exact harmonic superposition. In other words, a canon for three voices. More precisely, these last two investigations showed a greater relative importance of the seventh note (F-S cadence on the second period) and the eighth note (back to the A minor tonic) for each period. The latter once more prevailing over the former. That is, the perfect S-Q cadence on the sixteenth note prevailed over the preceding F-S cadence with the recovering of the initial tonality. The division of the period resulted in six semiquavers, one quaver, one crotchet (which meant relative lengths 1-1-1-1-1-1-2-4 with a 6:8 rhythm as shown in FIG. 1). The coherence of the melodic progression (wherefrom the observed regularity mainly proceeds) as well as the richness of the harmonic progression, the A minor tonality being accompanied with modulations in E minor (second bar), G minor (eighth bar), and F major (third and ninth bar) was apparent.

The first and seventh notes of each period fostered, respectively, adenine and thymine in third position; whereas, the third and eighth notes fostered in the same way cytosine and guanine. This confirmed the above division for the period and the relative lengths of notes. In other words, the seventh and eighth notes had lengths that were respectively twice and four times the first. This also showed that in an AT-rich environment strong beats were on the first and seventh notes, and therefore the measure bars were on the first. However, in a CG-rich environment the musical sequence started on an anacrouse (strong beat on the third and eighth notes, measure bar on the third).

The conclusion was that the protein had distinct metabolic roles, depending on its environment.

Actually, the range of its metabolic action was first demonstrated by the degree of its musical evolution. In comparison with the sequence of Euglena gracilis, in the three first measures an improvement of 56% of the melodic [regularity] level and of 16% of the harmonic [regularity] level was observed as defined from the minimization of the respectively melodic and harmonic distances between successive notes.

The search of musical similarities with other proteins showed the possibility to superpose cytochrome C onto endozepine with a musical reading frame compatible with the measure bar on the first note. This resulted in a slightly AT-rich molecule; thereby predicting an anti-depressive role for the cytochrome (and its music), through the eventual desinhibition of neurotransmission; as well as, a musical enchainment (then beginning on an anacrouse) with cytochrome oxidase. Cytochrome oxidase is slightly CG-rich and ends the respiratory chain.

As for tone quality, because tonality was present in A (minor), the quasi absence of the fourth (D) and the relative weakness of the fifth (E) compared to the distinct dominance of the tonic note and to the abundance of the octave (low A-medium A) privileged harmonics 1 and 2, to the prejudice of the followings, indicated an organ timbre with slightly different registers according to the passages.

As shown in FIG. 2, colors effectively grouped themselves into colored stains onto the mature protein with, as in the case for music, remarkable harmonic responses. The color determination was useful to confirm the musical decoding, insofar as some autocorrelations of notes were translated not into the musical period but in the spatial folding of the molecule. The spatial folding must eventually be subtracted to determine the musical periods. It was found that where a secondary peak of these autocorrelations, k=4, due to the α-helix of the beginning which can be seen in FIG. 2, corresponded to these foldings. Conversely, the musical decoding gave indications about the spatial structure of a protein.

### EXAMPLE 2

This example illustrates control of the decoding of a protein showing rhythmical variations. The decoding was controlled at different levels including the decoding of molecules known to be metabolically agonist and the coherence of the conclusions that were drawn from the musical similarities observed.

Recovering full sections of the metabolism facilitates the decoding. In Example 1, the “rhythmic formula” of cytochrome C was transcribed as follows:

|6/8 GDVEKGK:K:::|IFIMKCS:Q:::|CHTVEKG:G:::|, etc.
+ + + + + + + + + + + + + + + + + +

where the +underline the strong beats, the | indicate the place of measure bars and the: indicate the lengthening of notes.

In subunit III of cytochrome oxidase, which is musically chained to cytochrome C, the beginning is a four-time formula as shown by the internal similarities. The notes 7 to 22, which remind in their contours the manner of Bach, were split into groups of four notes, each one being superposable to the next. At the tenth measure, another measure which was not only superposable was found onto the first measure of cytochrome C, but was in fact, even practically identical to the third measure of the same cytochrome. This implied a lengthening of the eighth measure (as the cadence seen at the end of this measure already indicated in itself), in a six-time measure (FIG. 1):

```
|4/8 MTHQSHAY|HMVKPSPW|PLTGALSA|LLMTSGLA|
  + + + + + + + + + + + + + + + +
            MWFHFHSM|TLLMLGLL|TNTLTMYQ∥6/8 WWRDVTR:::::|
  + + + + + + + + + + + + + + + + + + +
            ESTYQGH:H:::|TPPVQKG:::::∥
            + + + + + + + + + + + +
```

This change in rhythm (from 4/8 to 6/8) was visible in base autocorrelations of the DNA where, at this point, the prominent peak went from the fourth to the sixth base triplet.

As seen in FIG. 1, the sequence started on an anacrouse emphasizing the strong beat on the third note, in view of the enchainment with the CG-rich rhythmic variant of cytochrome C.

### EXAMPLE 3

The example illustrates reconstitution of a metabolic chain including stimulations and inhibitions.

The decoding of histone 4 was particularly easy. The periodicity of 7 is clearly visible on the sequence at the outset of the molecule. The repetition of G within a two amino acid interval indicates a binary rhythm, and the GG cadences that end the two first periods specify right away a four-time rhythm:

|SGRGKGG:|KGLGKGG:|;
+ + + + + + + +

this pattern continued until the end of the sequence, with the only exception being the last measure which was syncopated to recover the rhythm of the first two measures. See FIG. 3. The global repartition of the notes showed a harmonic structure corresponding to the tone of a flute. The “skip of notes” repeated from the beginning, which suggested a sound with an attack and a timbre similar to that of Pan's pipes.

Histone 4 is one of the most conserved proteins among the animal and plant kingdoms. This does not mean that its metabolic action doesn't sometimes need to be tempered. The theme of histone 4's first two measures was found in inhibition and transposed to the fourth, in the conserved part of the beginning of chalcone synthase, which is the pigmentation enzyme of many flowering plants. See FIG. 3. This may be compared to the supposed role of chromatin, which histone 4 is part of, in the process of magnesium fixation. During spring, plants need a lot of magnesium for photosynthesis and the plant's fixation needs to be stimulated. Chalcone synthase is then inhibited; whereas, during the fall, the weaker stimulation of histone desinhibits chalcone synthase and allows the replacement of the green of the leaves by brighter colors of that season, the diversity of which, so much praised by the poets, becomes thus more understandable through their epigenetic component.

When listening to the musical transposition of histone 4, several auditors reported “an urge to eat chocolate” which contains magnesium. Some auditors found that “it produces the same effect as that of granulated magnesium, except that this effect is immediate in this case”. This presents some inconvenience for people having a slightly too high rate of cholesterol. Actually, the musical decoding of chalcone isomerase—the metabolically agonist of chalcone synthase, but which “works better” musically in stimulation—included a series of themes and variations whose succession reproduced, in flowering plants, themes of the full metabolic chain regulating cholesterol in man. In addition, the frequency of the ascending fourths in chalcone isomerase tended to approximate that observed in the alcali light chain of mammalian myosin, which stimulated muscular contraction (while magnesium acted as a muscular decontractant). Listening to the musical transposition of histone 4 encouraged physical exercise which is another way to lower cholesterol.

In fact, this example underlines the importance of a quasi-general phenomenon, that is, the epigenetic co-operation of different factors in the stimulation of protein synthesis, which accounts for the aspect meaningful in itself of the musical sequences. In this way for example, listening to myosin will generally suggest a military march.

### EXAMPLE 4

This example illustrates the biochemical analysis of an epigenetic cooperation involving harmonic superpositions. The biochemical analysis of these epigenetic cooperations is a valuable help for decoding.

Another way to stimulate epigenetically the muscular decontraction is heat, whose healing action for rheumatism, for example, is well known. The action of heat is conveyed by a group of proteins called heat shock, generally synthesized together. This suggests that the proteins should show harmonic superpositions. In fact, the hsp 27 protein, which appeared to be the most musical, superposed itself onto the beginning of the hsp 70 protein, the most abundant, which sort of played here the role of a bass line. These two molecules were again superposable together with the beginning of troponin C, which regulates calcium in muscular contraction. The conclusion was that it plays a role as an anti-rheumatic and that its musical level is high (FIG. 4). Other molecules, also of a high musical level and epigenetically sensitive, were implicated in this type of ailment, from the stimulation of prolactin and beta-lipotropin (precursor of beta-endorphin) to the inhibition of estrogen receptor, including the inhibition of IgE and interleukin 1 beta.

These examples clearly show how large sections of the metabolism can be reconstituted step by step, with many ways to check or control the coherence of the results obtained, and thereby to precise the musical decoding of the concerned proteins.

### EXAMPLE 5

This example shows a practical application of the method of this invention using the transcriptions in the form of either musical scores, or of recordings of the obtained musical sequences.

The recordings of musical sequences may be realized from musical scores described earlier, by using one of the methods evaluated in B. H. Repp, J. Acoust. Soc. Am. 88, p.622 (1990). The most precise of these methods was used in the examples hereby given.

In the fields of agronomy and textile industries this invention provides methods to stimulate certain specific protein synthesis, for example, bovine lactation, fermenting of baker's yeast, the sweet taste of some fruits, animal or plant fibres (keratine of sheep's wool, fibroin of silkworm, etc.), as well as the proteins specific to certain medicinal plants. In the field of environment the method of this invention is used, for example, in the assimilation of industrial effluents through plants by stimulating the biosynthesis of the corresponding proteins.

The method of this invention was used on a cow who regularly, during 15 days and at the time of milking, listened to recordings of musical transcriptions of the amino acid sequences of bovine prolactin, lactoglobulin, and lactalbumin. A reduction, by a ratio of 3, of the relative quantity of whey was observed, resulting in a milk highly enriched in proteins, and in a particularly savory cheese.

In another experiment growing tomato plants were given a “cocktail” of musical transpositions of different proteins including: specific virus inhibitors, various extensions, then a flowering enzyme (LAT 52), an antibacterial protein having musical similarity to thaumatin, an improvement of sugar percentage (P 23), and inhibitors of fruit softening enzymes (pectinesterase and polygalacturonase). A distinct increase in size and number of fruits (summing up to a ratio of about 3.5) was observed, as well as, a sensitive increase of the sweet taste in a significant proportion of the fruits that had particularly received P 23.

These noteworthy results go along with a certain amount of precautions, namely, there exist some counter-indications to an excess of stimulation, especially of prolactin, which must be cautiously taken into consideration by breeders that carry out these methods, as well as for the animals themselves who may be fragilized. In the experiments carried out on cows with Mozart music—bovine prolactin has in fact, apart from a “musical level” particularly high which can here define in a mathematically simple way some musical turns that can be qualified as “typically Mozartian”—the rate of mammites could seem worrying. In such a case one ought to complete the hearing of prolactin with that of alpha-1 antitrypsin, whose musicality is also very elaborate and whose metabolism is complementary. Similarly for tomatoes receiving outside stimulations, one must be cautious not to interrupt the cycle too suddenly.

These results give an indication of the order of magnitude of results obtainable in such conditions.

### EXAMPLE 6

In the therapeutic and preventive fields, many ailments are characterized by a specific metabolic weakness and can therefore be efficiently prevented or treated with the help of the present invention. This example illustrates such prevention or treatment.

Because the minimal length of a musically active sequence is of the order of that of a signal peptide, i.e., from several amino acids to a few tens, this action may be very fast and appear after a few seconds or a few minutes. Nevertheless, the complete integration of the produced effect can take slightly more time, or even require, in case of a strong cultural conditioning, i.e., a certain initial training. But usually, this initial training is accomplished rather rapidly for the obvious benefit of the persons concerned.

For a responsible use of the described method, it is important to know the metabolic role of the molecules involved. And it is of course one of the interests of the musical decoding of proteins (associated to the corresponding colors) to allow, by systematically spotting the similarities and counter-similarities of melodies (and colors) from the protein sequences that are known and available in data banks, to select proteins that are metabolically agonist and antagonist of a given protein, for which the degree of musical elaboration also gives an indication of the importance of its metabolic role. The described method therefore allows determinations of precise particular indications for some proteic sequences.

As earlier noted, in animal or plant proteins, especially among the most musical ones, successive melodic fragments of human metabolic chains were observed. Therefore, the transpositions which were found to be active on man were not limited to human molecules. On the other hand, the metabolism of those species seems in some way more “specialized” for the production of certain molecules, and it is indeed the most musical proteins that will be the most important for the applications. Of course, these correspondences between different species facilitate the delimitation of the metabolic role, and the decoding of proteic sequences.

The musicality of a molecule implies in itself that its epigenetic stimulation is preferable for a therapeutic use, (because of the range of its metabolic interactions), to its direct absorption. The “most musical” molecules are generally those for which either the production by genetic engineering, or the therapeutic use which derives from it, will meet some problems, such as of transportation to the site of action, or of stability, or more specifically of secondary effects related to doses that should be much more important than what they are in the body to obtain comparable effects, because then, the scaling waves naturally associated to their production are not present any more. This is particularly true for the inhibition of proteins, when the natural inhibitor is much heavier, or simply when the production needs to be reduced at a given time or in a systematic way.

Eventually, concerning the use of transcriptions of proteic sequences, the very quickness of their action may allow, by differential comparison, especially bipolar, of their positive and negative effects to precisely which one is the most appropriate in a given situation. This identification is facilitated by the comparison with transcriptions of known proteic sequences of acoustic or electromagnetic phenomena exhibiting distinct series of frequencies, and for which some effects have been observed in a similar situation.

As will be appreciated from the above, the invention is in no way limited to those methods of putting it into effect, of construction and of application which have been described above in detail; on the contrary, it covers all versions which may be conceived of by workers skilled in the art, without exceeding, either the framework or the scope of the present invention.

That which is claimed is:

1. A method of regulating protein synthesis in situ comprising:
(a) determining the sequence of musical notes associated with the amino acid chain of a protein by associating with each amino acid a musical note whose frequency is transposed from the proper frequency of the amino acid;
(b) determining the musical periods of said sequence of musical notes by identifying similar series of musical notes;
(c) comparing the repartition of said musical sequence of said amino acid chain to the average repartition of said musical notes of the whole of proteins so as to determine the tone quality; and
(d) regulating the biosynthesis of said protein by playing said sequence of musical notes, including the musical period of said notes and the tone quality of said musical notes.

2. The method of regulating protein synthesis according to claim 1 further comprising:
determining the lengths of said musical notes by rectifying collectively, and then rectifying individually said musical periods by adjusting the phrasing to the measure of said musical sequence.

3. The method of regulating protein synthesis according to claim 1 further comprising determining the frequency of said musical note according to a code comprising:
(a) taking the frequency of each amino acid in its free state, proportional to its mass,
(b) minimizing the global harmonic distance between the frequencies of each pair of amino acids in said protein while taking into account the proportion of each amino acid in the population of transfer RNAs within a cell where synthesis of said protein takes place, and wherein the displacement of the note frequency towards its synchronized value is inferior to half the interval between the two synchronized frequencies which surround said keynote frequency, then
(c) transposing the frequencies thus obtained into the auditive range, said code being relative to the biosynthetic stimulation of said protein; and
(d) obtaining said code relative to its inhibition by symmetrization of the logarithms of heretofore obtained frequencies with respect to their central value considered as the origin.

4. The method of claim 3, wherein said code comprises the following notes of the chromatic tempered scale in ascending order:
Gly=low A; Ala=C; Ser=E; Pro, Val, Thr, Cys=F; Leu, Ile, Asn, Asp=G; Gln, Lys, Glu, Met=A; His=B flat; Phe as well as SeC=B; Arg, tyr=sharp C; Trp=sharp D.

5. The method of claim 3, wherein said code comprises the following notes of a chromatic tempered scale, in ascending order:
Trp=C; Arg, Tyr=D; Phe as well as SeC=E flat; His=E; Gln, Lys, Glu, Met=F; Leu, Ile, Asn, Asp=G; Pro, Val, Thr, Cys=A; Ser=B flat; Ala=Sharp D; Gly=sharp F,
which are deduced from the notes of the code by taking the notes of the chromatic tempered scale which are symmetrical to those of said keynotes with respect to central G.

6. The method according to claim 3 wherein said synthesis stimulates synthesis of a protein in a plant.

7. The method according to claim 1, wherein each sound transposition of quantum vibrations associated with the biosynthesis of a given protein is completed by the color transposition of quantum vibrations associated to the mature protein after it is spatially folded back over itself, according to a code specific to the stabilization of that protein or to the inhibition of its biosynthesis obtained through the musical sequence realized according to claim 1, which code is deduced from the code obtained from claim 1, by application of the formula ν≡ν₀ Argch (e^{ƒ/ƒ}₀)^{Logch 1}, where ƒ, ƒ₀ are the musical frequencies and ν, ν₀ the frequencies of colors, with the index 0 showing the central values.

8. The method according to claim 6, wherein the stabilization of proteins stimulated by the musical sequences obtained according to claim 1 consists in the association to the different amino acids of the following colors:
Gly=dark red; Ala=bright red; Ser=orange; Pro, Val, Thr, Cys=ochre; Leu, Ile, Asn, Asp=lemon yellow; Gln, Glu, Lys, Met=green; His=emerald; Phe=blue; Arg, Tyr=indigo; Trp=purple

9. Transcriptions of a musical sequence according to claim 1 selected from the group consisting of musical scores of said musical sequence and audio recordings of music according to said musical sequence.

10. The method according to claim 9 for the characterization of proteic sequences fit to be regulated by using any of the transcriptions characterized in that one delimits their metabolic role by decoding with the method according to claim 3, thereby showing the musical similarities and anti-similarities that they present with other proteins, the harmonic superpositions with other proteic melodies, or a combination of these factors, from which the agonisms and antagonisms can be deduced.

11. The method according to claim 9, in which the characterization for a given application is refined by bipolar differential comparisons with the positive or negative effects obtained by using said transcriptions.

12. The method according to claim 10, in which the characterization for a given application is refined by identification, through musical similarity or anti-similarity of the proteins involved during positive or negative effects due or associated to acoustic or electromagnetic phenomena exhibiting distinct series of frequencies.
